(19) United States
(12) Patent Application Publication
(10) Pub. No.: US 2021/0237131 A1
(43) Pub. Date: Aug. 5, 2021

(54) METHOD AND APPARATUS FOR CLEANING A MAT

(71) Applicant: Steve SUBAR, Chicago, IL (US)

(72) Inventors: Steve SUBAR, Chicago, IL (US); Mario PENA, Chicago, IL (US); Tyler GRUDOWSKI, Orland Park, IL (US); Angelica CZARNECKA, Chicago, IL (US); Yuri GALLEGOS, Plainfield, IL (US)

(73) Assignee: Steve SUBAR, Chicago, IL (US)

(21) Appl. No.: 17/168,103

(22) Filed: Feb. 4, 2021

Related U.S. Application Data
(60) Provisional application No. 62/970,081, filed on Feb. 4, 2020.

(51) Int. Cl. B08B 13/00; B08B 3/08; B08B 3/04; B08B 3/02; B08B 5/04; A63B 71/00; B65G 43/08; B65G 39/10; A61L 2/10

(52) U.S. Cl. CPC B08B 13/00; B08B 3/08; B08B 3/041; B08B 3/022; B08B 5/04; B65G 23/06; B65G 43/08; B65G 39/10; A61L 2/10; B08B 2203/027; B65G 2203/0233; A63B 71/0036

(57) ABSTRACT

Methods and apparatus for cleaning a mat are disclosed. The disclosed process receives a user identifier, such as a phone number and/or PIN, at a mat cleaner from a user. The mat cleaner then accepts a mat, such as a yoga mat, form the user and conveys the mat through the mat cleaner. Various guides and sensors are employed to facilitate straight insertion and smooth conveyance of the mat through the mat cleaner. As the mat is conveyed, the mat is illuminated with ultraviolet light and sprayed with a recirculated cleaning solution. The mat cleaner includes a sensor, such as a float switch, to facilitate determination of the circulated cleaning solution level.

The present disclosure relates in general to processing mats, and, in particular, to methods and apparatus for cleaning a mat using a mat processing device.

### BACKGROUND

Traditionally, users of exercise mats, such as yoga mats, clean and disinfect their mats manually. For example, after a yoga session, a participant may spray his/her mat with a cleaner and wipe the mat down with a towel. However, this method is cumbersome and often ineffective. Mat processing devices have been used to attempt to overcome these limitations of manual mat cleaning. However, current mat processing devices, and the associated methods of using these mat processing devices, lack certain improvements disclosed herein. For example, current mat processing devices suffer from jammed mats. In addition, current mat processing devices do not sufficiently and efficiently clean the mats they process.

### SUMMARY

Methods and apparatus for cleaning a mat are disclosed. The disclosed process receives a user identifier, such as a phone number and/or PIN, at a mat cleaner from a user. The mat cleaner then accepts a mat, such as a yoga mat, form the user and conveys the mat through the mat cleaner. Various guides and sensors are employed to facilitate straight insertion and smooth conveyance of the mat through the mat cleaner. As the mat is conveyed, the mat is illuminated with ultraviolet light and sprayed with a recirculated cleaning solution. The mat cleaner includes a sensor, such as a float switch, to facilitate determination of the circulated cleaning solution level.

### BRIEF DESCRIPTION OF THE DRAWINGS

FIG. 1 is a side view of an example mat cleaner.

FIG. 2 is another side view of the example mat cleaner with the door of the mat cleaner in an open position.

FIG. 3 is yet another side view of the example mat cleaner highlighting the drive train of the mat cleaner.

FIG. 4 illustrates a top view of the example mat cleaner.

FIG. 5 illustrates a rear view of the example mat cleaner.

FIG. 6 is a flowchart of an example process for cleaning a mat using the mat cleaner.

### DETAILED DESCRIPTION OF THE PREFERRED EMBODIMENTS

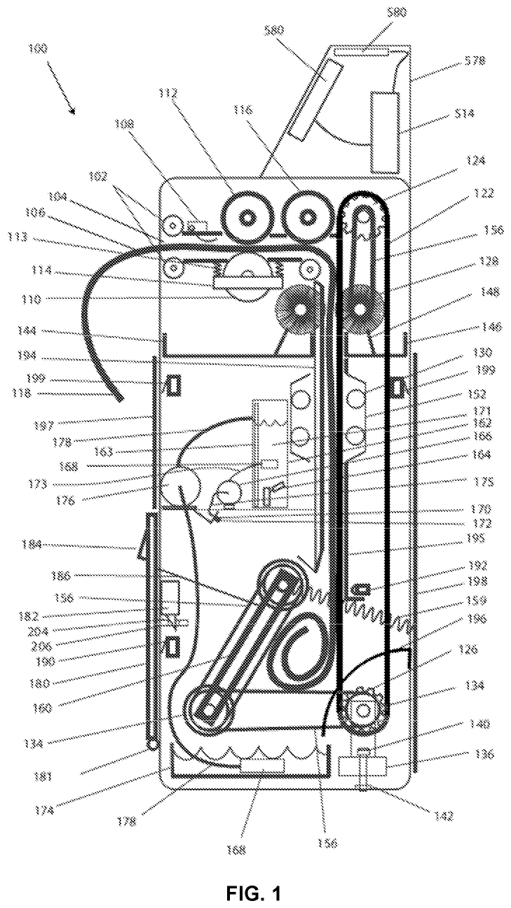

FIG. 1 illustrates a side view of an example mat cleaner 100. In this example, the mat cleaner 100 includes one or more entrance guide rollers 102, which are located at or near the top and bottom of an insertion slot 104. The entrance guide rollers 102 rotate upon contact with a mat 106 to allow the mat 106 to be inserted into the insertion slot 104 without getting stuck on a surface of the mat cleaner 100 due to friction.

Once the mat 106 contacts one or more activation switches 108 located near the front of the insertion slot 104, the mat cleaner 100 is activated, thereby causing one or more idler rollers 110 and one or more front driver rollers 112 to pull the mat 106 into the mat cleaner 100. The activation switches 108 are structured to prevent the mat 106 from being inserted in a crooked orientation. Ends of the idler roller 110 are connected to one or more spring loaded tensioner brackets 114, allowing the idler roller 110 to dynamically apply a predefined pressure to convey the mat 106 in accordance with dimensions of that particular mat 106. One or more rear driver rollers 116 push the tail end 118 of the mat 106 into a conveyor subsystem 120.

The conveyor subsystem preferably includes one or more vertically aligned conveyor chains 122 and/or belts that are threaded around one or more upper sprockets 124 and one or more lower sprockets 126. The upper sprockets 124 and lower sprockets 126 cooperate to move the conveyor chains 122. When a mat 106 contacts the conveyor chains 122, the movement causes the mat 106 to be conveyed vertically in the mat cleaner 100 through one or more brushes 128 and/or UVC lamps 130.

A bottom shaft 132 of the conveyor subsystem 120 houses the lower sprockets 126 and pulleys 134, which are installed into a main tensioner bracket 136. The main tensioner bracket 136 is preferably mounted to a frame using chain tensioner bolts 140. Tightening down these chain tensioner bolts 140 effectively causes the height of the main tensioner bracket 136 to change, which determines a conveyor chain tension. Once the conveyor chains 122 are properly tensioned, a nut 142 is preferably placed on the ends of the chain tensioner bolts 140 to help ensure that the chain tensioner bolts 140 will not loosen and cause the main tensioner bracket 136 to move. Because the main tensioner bracket 136 may be subjected to significant stress, support measures are preferably included to stabilize the main tensioner bracket 136, such as L-brackets, thick shims that fasten the bracket to the frame, and/or any other suitable bracket stabilization means.

The brushes 128 (e.g., roller brushes) preferably remove dirt and/or debris from both sides of the mat 106 substantially simultaneously. The dirt and/or debris may then be collected in a front dust bin 144 and/or a rear dust bin 146 after being removed from the bristles of the brushes 128 via brush combs 148.

A UVC system includes one or more UVC lamps 130 to illuminate the mat 106 with the radiation necessary to eradicate pathogens. The pathogens are preferably eradicated on both sides of the mat 106 simultaneously. One or more UVC lamp covers 152 house the UVC lamps 130 along and prevent harmful radiation from affecting users of the mat cleaner 100. There exists a reflective surface on the inside of the lamp covers in order to magnify the UVC light dosage that is applied to the mat.

When a mat 106 exits the conveyor subsystem 120, the mat 106 hits a first series of round belts 156 that make up a rolling subsystem 158. The round belts 156 preferably sit in grooves of adjacent pulleys 134 that are preferably located on an inner door arm 160, and a lower conveyor shaft. The round belts 156 make contact with rubber bands that sit in the grooves of the pulleys 134 to prevent slippage of the round belts 156. The round belts 156 can be made of varying lengths, materials (polyurethane rubber, rubber, etc.), and designs (round belts, timing belts, etc.). The round belts 156 are preferably of a suitable flexibility, while still maintaining an ability to rotate due to the drive chain control. A person of ordinary skill in the art will readily appreciate that compatible pulleys and/or sprockets may be selected for different belt types and lengths

As the pulleys 134 and round belts 156 rotate, the mat 106 is pulled along the first set of round belts 156 to the second set of round belts 156 located on an inner door arm 160 and a rolled mat 106 begins forming. The rolled mat 106 continues increasing in diameter as the mat 106 continues being fed through the mat cleaner 100. Due to a spring mechanism of the inner arm door 160 and flexibility of the round belts 156, the angle of the inner arm door 160 is able to adjust to the increasing mat thickness while keeping a tight roll.

A hydraulics subsystem preferably sprays both sides of the mat 106 with an all-natural cleaning solution. A HOCL compatible reservoir 162 houses the cleaning solution. The reservoir 162 is composed of fittings for the inlets, a fitting for the outlet, a fluid level sensor 163, and a lid. All of these components are preferably connected to the reservoir 162 with a watertight seal. For example, a thread seal, O-rings, nuts, or any combination thereof. The reservoir 162 preferably includes an inlet for air to prevent any pressure build up. This can be accomplished with an air inlet fitting and/or holes located on the reservoir lid. Additionally, an electronic float switch 164 located inside the reservoir 162 may automatically detect if the cleaning solution needs to be refilled. This data is preferably transmitted to a cloud-based application to notify one or more entities to refill the cleaning solution 171.

Electronically controlled spray pumps 166 extract the cleaning solution 171 from the reservoir 162 through a nozzle strainer 168 to prevent small particles from clogging the spray nozzle 170 openings. For example, the strainer 168 may be a mesh bag and/or a disk filter. The strainer 168 may be located inside of the reservoir 162 or the strainer 168 may be directly connected in a line before the solution reaches the spray nozzles 170. The filtered cleaning solution 171 travels from the spray pump inlet 173, to the spray pump outlet 175, to the spray nozzles 170 through a series of tubes that are connected using fittings, such as but not limited to john guest fittings. The cleaning solution 171 then exits the spray nozzle 170 openings and is sprayed onto the mat 106 to serve as a secondary sanitizing method. The spray nozzles 170 are preferably installed at a predetermined height, location, and angle, so that the spray nozzles 170 spray both sides of the mat 106 when the mat 106 is being rolled. A cone 172 of the spray nozzles 170 may have a predetermined angle that allows some or all of surface of the mat 106 to be sprayed with a suitable spray amount.

A drip tray 174 preferably collects some or all of any overspray that occurs. The water pump 176 pulls this overspray through the strainer 168 and into a recirculation line 178. The strainer 168, which may be a mesh bag and/or a disk filter, prevents debris from being introduced into the hydraulics system. The recirculation line 178 preferably sits predominantly flat on the drip tray 174 in order to suction a sufficient quantity of overspray. This can be accomplished by fastening the recirculation line 178 (e.g., with tape and/or a fitting) to the drip tray 174. Alternatively, the recirculation line 178 can be kept relatively flat by weighing it down (e.g., with a heavy plate or rocks). The filtered overspray is then funneled back into the reservoir 162 through the recirculation line 178. Recirculating the cleaning solution 171 allows the hydraulic subsystem 177 to operate as a maintenance free closed loop.

The door 180 preferably has material etched out, so light is visible through the front in order to draw attention to the mat cleaner 100 to maximize utilization. The etch can be of the logo, any shapes, or lettering to provide users with visibility that the UV light 130 is on. If this etch is located on a panel that allows spray to exit the mat cleaner 100, the etch is preferably covered with a clear backing.

The door 180 preferably includes lock brackets on each side that interact with solenoid locks 182 to keep the door 180 closed. For safety reasons, the door 180 on hinges remains locked until a cleaning cycle is complete, so hands are not inserted into the mat cleaner 100 when it is operating. A handle 184 located on the door 180 enables a user to open the door 180. A steel cable 186 connects the inner door arm 160 to the door 180 allowing for both the inner door arm 160 and the door 180 to open automatically, creating an unobstructed opening for mat 106 retrieval.

An electronics control box 188 preferably includes a printed circuit board (PCB), microprocessor, input devices (e.g., sensors, microswitches, touch screen display, etc.) and output devices (e.g., relays, motor, pumps, lights, touch screen display, etc.) to control operation of the mat cleaner 100. The PCB supports and electronically controls the electro-mechanical components that make up the mat cleaner 100. Additionally, the PCB may gather and record data from the input signals it receives. The PCB preferably includes a microcontroller and software and connects to a Wi-Fi network and a cellular network.

A door switch 190 located between the door 180 and the frame is able to determine whether the door 180 is opened or closed. When the door 180 is open, and not making contact with the door switch 190, the mat cleaner 100 may prompt an error message indicating the door 180 is open. Similarly, if the door switch 190 detects the door 180 has not been opened after a completed cleaning cycle, the user may be warned that a mat 106 remains in the mat cleaner 100.

Aside from controlling the systems within the mat cleaner 100, the electronics control box 188 may include a cellular antenna that allows the mat cleaner 100 to communicate with a cellular network. The cellular connection allows for pushing firmware updates over the air along with reporting any data pertaining to usage or faults.

When a mat 106 is conveyed through the mat cleaner 100 and the mat 106 passes an optical sensor 192, the hydraulics subsystem 177 is activated. Similarly, the hydraulics subsystem 177 is deactivated when the tail end of the mat 106 passes the optical sensor 192. If the optical sensor 192 does not detect a mat 106 after a predetermined amount of time once the cleaning cycle has begun, the mat cleaner 100 may indicate a mat jam and the mat 106 may be automatically reversed.

The mat cleaner 100 preferably includes a front plate 194 and a back plate 195 to keep the mat 106 flat when the mat 106 is being conveyed through the mat cleaner 100. The front plate 194 and the back plate 195 are preferably structured to reduce the amount of surface friction and unnecessary openings in locations where a mat 106 may make contact with the plates 194, 195. The low friction plate helps prevent a mat 106 from jamming by providing a low friction, curved surface for the mat 106 to be conveyed along.

Guide round belts 156 cause a horizontally inserted mat 106 to curve downwards and contact the vertical conveyor subsystem. One or more sprocket covers 196 help prevent a mat 106 from getting stuck between the conveyor chains 122 and the teeth of the lower sprockets.

In addition to acting as an external body of the mat cleaner 106, the front cover 197 and the rear cover 198 may be used as maintenance access panels. When a panel is removed, contact switches 199 located behind the front cover 197 and/or the rear cover 198 preferably deactivate. This may prompt an error message along with disabling both the motor and the UVC lamps.

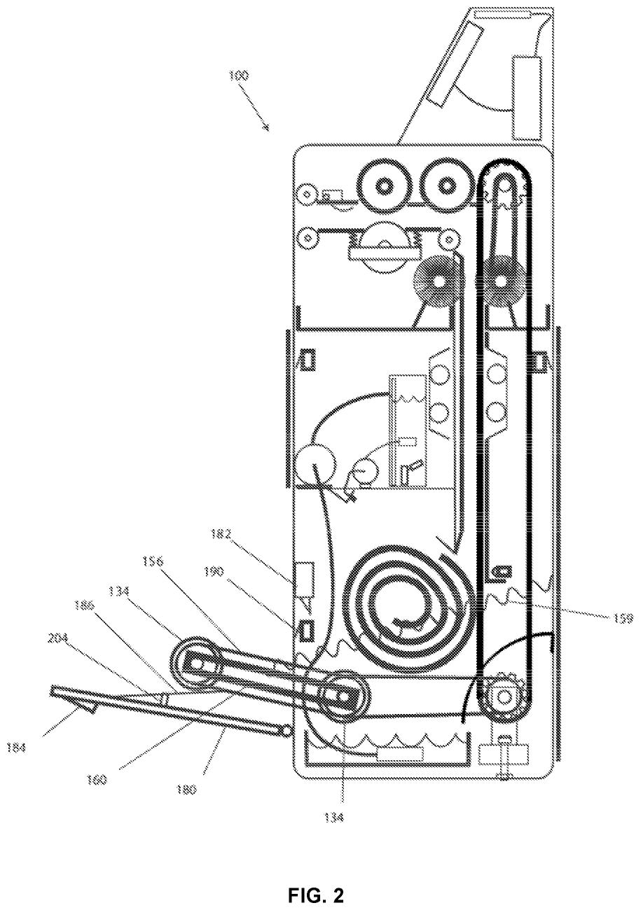

FIG. 2 illustrates another side view of the example mat cleaner 100 with the door of the mat cleaner 106 in an open position. In this example, the inner door arm 160, made up of one or more pulleys 134 and one or more round belts 156, causes the mat 106 to roll up tightly.

Once a cleaning cycle is complete, one or more solenoid locks 182 deactivate and retract from an opening 204 in the lock brackets 206. This retraction causes the door 180 to open automatically, so users know where to remove the mat 106 from. A handle 208 enables users to open the door 180. A steel cable 210 connects the inner door arm 160 to the door 180 allowing the inner door arm 160 and the door 180 to both open automatically, creating an unobstructed opening for mat retrieval.

The door switch 212 located between the door 180 and the frame 214 is able to facilitate determination of whether the door 180 is opened or closed. When the door 180 is open, and not contacting the door switch, the mat cleaner 100 preferably generates an error message indicating the door 180 is open. Similarly, if the door switch 212 indicates that the door 180 has not been opened after a completed cleaning cycle, the mat cleaner 100 preferably warns the user that there a mat 106 remains in the machine.

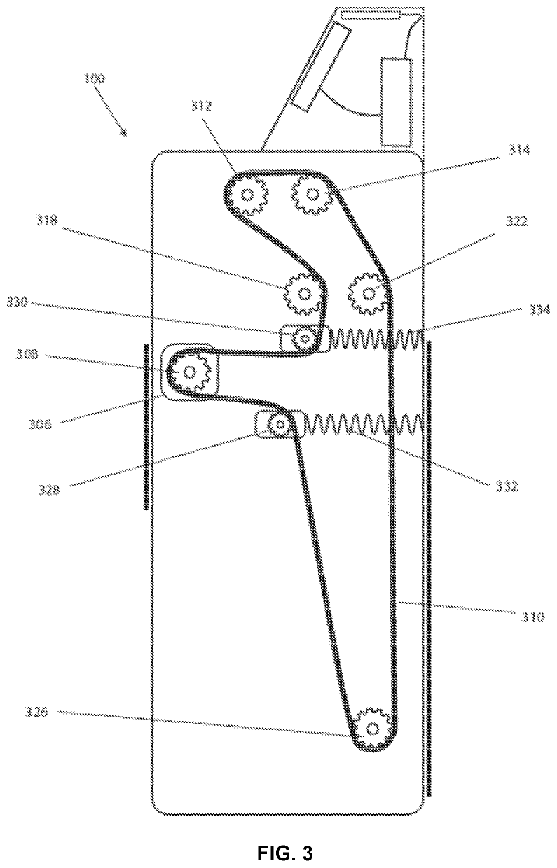

FIG. 3 illustrates yet another side view of the example mat cleaner 100 highlighting the drive train 302 of the mat cleaner 106. Once the activation switches 108 are engaged by contacting a mat 106, a stepper motor 306 is activated. The motor sprocket 308 installed on the shaft of the stepper motor 306 controls the drive chain 310 that drives the system. The drive chain 310 is threaded around the front drive sprocket 312 and the rear drive sprocket 314, which control the driver rollers 316. The drive chain 310 is also threaded around a front brush roller sprocket 318, which controls the front brush 320. The drive chain 310 s also threaded around a rear brush roller sprocket 322, which controls the rear brush 324. The drive chain 310 is also threaded around the mat rolling mechanism sprocket 326, which controls the rolling of the mat 106 along with driving the conveyor system.

The drive chain 310 is also threaded around the teeth of a forward tensioner sprocket 328 and a reverse tensioner sprocket 330. The forward tensioner sprocket 328 is connected to a forward tension spring 332 and the reverse tensioner sprocket 330 is connected to a reverse tension spring 334. These spring mechanisms allow the forward tensioner sprocket 330 and the reverse tensioner sprocket 330 to move dynamically and adjust chain tension based on torque applied to convey the mat 106, mat dimensions and mat material. An alternative method, effectively allowing the drive chain 310 to handle changes in the tension, employs tensioner sprockets placed between two sides of the drive chain 310. In this embodiment, the tensioner sprockets help prevent the drive chain 310 from jumping teeth when tension changes occur.

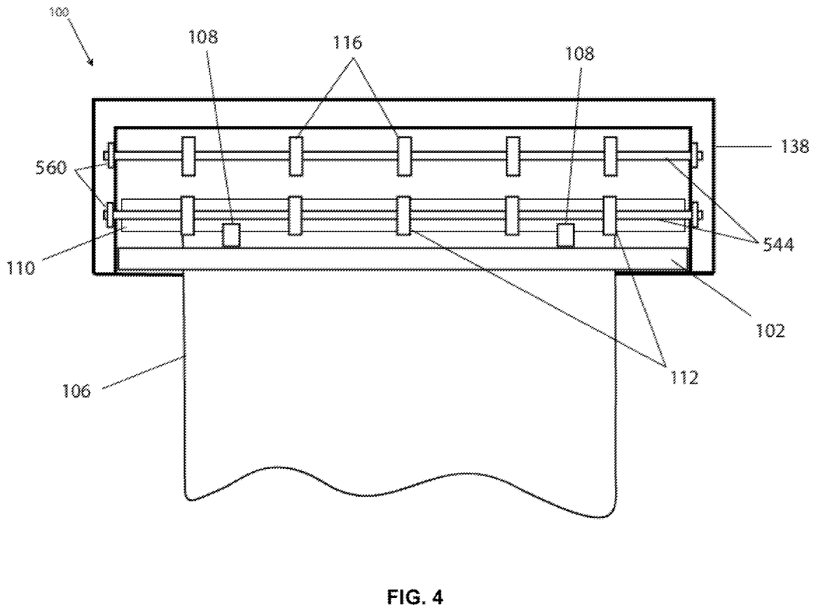

FIG. 4 illustrates a top view of the example mat cleaner 100. The driver rollers 316 exist on shafts located within the housing of the mat cleaner 100. The end of each shaft is installed into a flange bearing 402 to allow for a smooth rotation when driven by the drive chain 310. The driver rollers 316 can have a flat or grooved surface and they can be made of any high friction material, such as rubber. The driver rollers 316 can either be made up of multiple individual traction rollers or one solid roller.

Once a mat 106 contacts one or more activation switches 108 located near the front of the insertion slot 404, the mat cleaner 100 is preferably activated causing an idler roller 406 and front driver roller 316 to pull the mat 106 into the mat cleaner 100. The activation switches 404 are structured to prevent the mat 106 from being inserted in a crooked orientation.

The rear driver rollers 316 push the tail end of the mat 106 to the conveyor subsystem 120. The idler roller 406 is preferably a solid pipe that is able to rotate freely and is preferably be made of a material with a low friction coefficient, such as PVC. The idler roller 406 may have a mechanism in place that allows the idler roller 406 to automatically adjust the idler roller height for any different mat thicknesses.

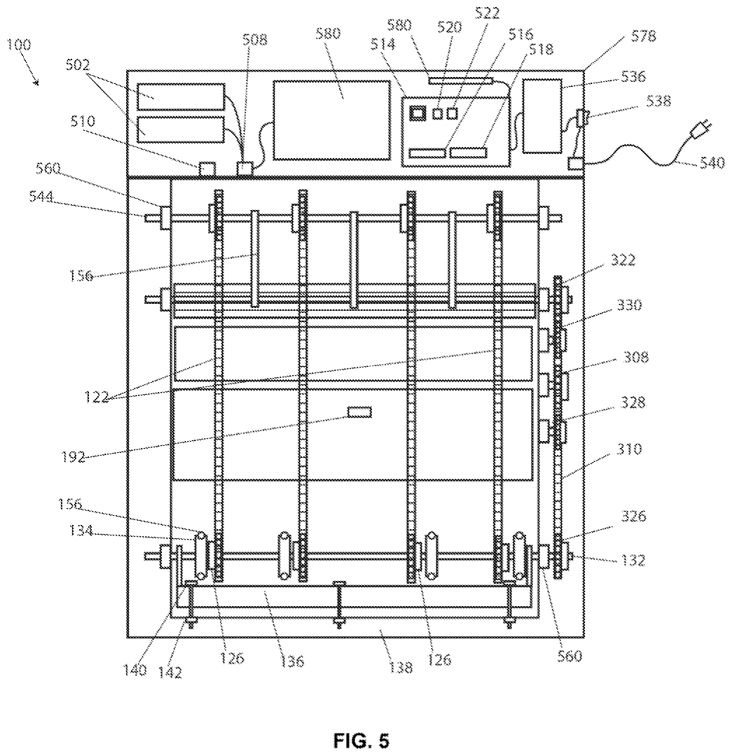

FIG. 5 illustrates a rear view of the example mat cleaner 100. The UVC Lamp Ballast 502 is an electronic device used to energize the lamp coils to vaporize mercury amalgam inside the lamp coils and excite the associated atoms to produce UVC light preferably in the 230 nm frequency typical of UVC light. UVC Lamp Ballast 502 preferably works with 120 VAC. The mat cleaner 100 detects when a lamp 130 is faulty and triggers a UVC lamp ballast failure event/error.

The ballast relay 508 is used to control the 120 VAC lamp ballasts, preferably with a 24 VDC output from the PCB 514. The spray pump relay 510 is preferably used to activate one or more 120 VAC pumps with a 24 VDC output from the PCB 514. The electronics control box 188 controls a PCB 514, microprocessor, input devices (sensors, microswitches, touchscreen) and output devices (relays, motor, pumps, lights, screen).

The PCB 514 supports the electro-mechanical components that make up the mat cleaner 100. The PCB 514 consists of input ports 516, output ports 518, an SD 520, and a SIM card 522. The input ports 516 are connected to sensors 518 and microswitches 520 that provide information to the PCB 514 regarding their current state. The PCB 514 can transmit data from the input ports 516 to a cloud-based application for automated metrics reporting and quality control. The output ports 518 control the sprayer pumps 524, the water pump 526, the lamps 528, the door locks 530, and the motor 532. The SD card 520 houses images that are displayed on a touch screen 534. The SIM card 522 is preferably specific to each individual mat cleaner 100 and allows the mat cleaner 100 to communicate over the air via a cellular network.

A power supply 536 is a device used to power the electronics by preferably converting 120 VAC to 24 VDC. A power button 538 is preferably a manual switch to power the mat cleaner 100 on. A power plug 540 is a cable that connects the mat cleaner 100 to an electrical outlet.

Each sprocket 542 controls a shaft 544 of varying functionality, so the drive chain length and sprocket sizes of the drive chain system are predetermined to have an appropriate rpm. The drive chain 310 is powered by the motor 546 and controls the brushes 548, the driver rollers 550, the conveyor subsystem 120, and the rolling subsystem 554.

The ends of the shafts 544 that house upper sprockets 556 and lower sprockets 558 are installed into flange bearings 560 to allow for a smooth rotation. Flange bearings 560 are present throughout the mat cleaner 100 and provide the shafts 544 a frictionless surface to rotate in. Sprocket covers 562 prevent a mat from getting stuck between the conveyor chain 564 and the teeth 566 of the lower sprockets 558.

The conveyor chains 564 are threaded around the upper sprockets 556 and lower sprockets 558, causing them to rotate when the drive train system 568 is activated and engages the rolling mechanism sprocket 569. The pulleys 134 and round belts 156 on the lower shaft 570 of the conveyor subsystem 120 are part of the rolling subsystem 574.

When the mat 106 is conveyed through the mat cleaner 100, and the mat 106 passes the optical sensor 192, the hydraulics subsystem 177 is activated. Similarly, the hydraulics system 177 is deactivated when the tail end of a mat 106 passes the optical sensor 192. If the optical sensor 192 does not detect a mat 106 after a predetermined amount of time once the cleaning cycle has begun, this may indicate a mat jam, and the mat 106 may be automatically reversed.

Aside from controlling the systems within the mat cleaner 100, the electronics control box 578 includes a cell antenna 580 that allows the mat cleaner 100 to communicate with the cellular network. The cellular connection allows for pushing firmware updates over the air along with reporting any data pertaining to usage or faults.

The electronics controls box 578 includes a touch screen display 580 which allows users to interact with the mat cleaner 100. For example, the touch screen display 580 can be an iPad, Android, or any other suitable screen. No credit card, barcode scan, or cash is necessarily needed to activate the mat cleaner 100 or pay for cleans. Instead, users may enter their phone number and pin on the touch screen display 580. Additionally, on the touch screen display 580, the user may see updates about the progress of the cleaning cycle and/or targeted ads. A maintenance screen 582 and an admin screen 584 may also be accessed via the touch screen display 580 with a series of predestined clicks and/or a secret password preferably only available to certain individuals. The maintenance screen 582 allows an individual to manually control certain machine functions such as forward, reverse, priming, recirculation, and unlocking the door. The admin screen 584 allows an individual to put a mat cleaner 100 in a test start mode, see the firmware version, flash a USB drive, etc.

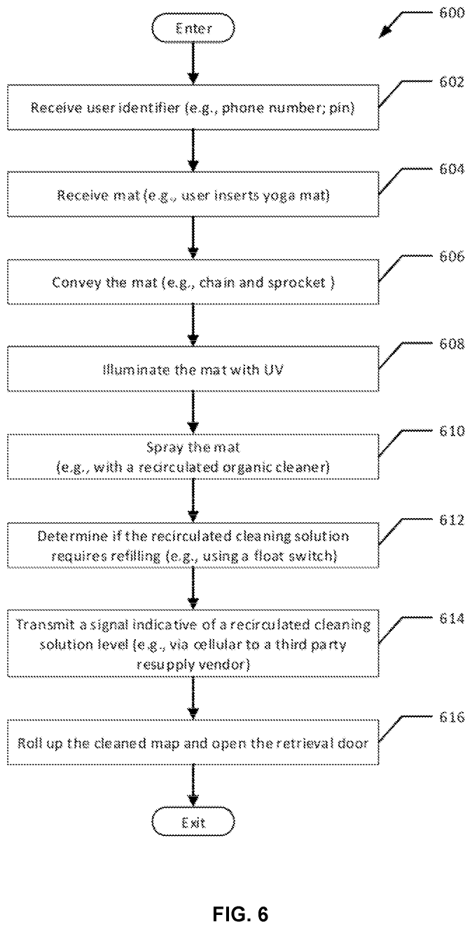

FIG. 6 is a flowchart of an example process 600 for processing mats. Although the process 600 is described with reference to the flowchart illustrated in FIG. 6, it will be appreciated that many other methods of performing the acts associated with process 600 may be used. For example, the order of many of the operations may be changed, and some of the operations described may be optional.

In this example, the process 600 begins by receiving a user identifier. For example, a user may enter a phone number and/or a personal identification number (PIN) (block 602). The mat cleaner 100 then receives the mat 106 (block 604). For example, the user may insert a yoga mat. The mat cleaner 100 then conveys the mat 106 (block 606). For example, a chain and sprocket conveyance system may be used. The mat cleaner preferably 100 removes dirt/debris with brushes. The mat cleaner 100 then illuminates the mat 106 with ultraviolet radiation (UV) (block 608). During conveyance, the mat cleaner 100 also sprays the mat 106 with a cleaner (block 610). For example, the mat cleaner 100 may spray the mat 106 with a recirculated organic cleaner. The mat cleaner 100 also determines if the recirculated cleaning solution requires refilling (block 612). For example, a float switch may be used to facilitate determining a fluid level of the recirculated cleaning solution. The mat cleaner 100 may transmit a signal indicative of the recirculated cleaning solution level (block 614). For example, the recirculated cleaning solution level may be transmitted via cellular to a third party resupply vendor. After the mat 106 is cleaned, the mat cleaner 100 preferably rolls up the cleaned mat and automatically opens the retrieval door (block 616).

In summary, persons of ordinary skill in the art will readily appreciate that methods and apparatus for cleaning a mat have been provided. The foregoing description has been presented for the purposes of illustration and description. It is not intended to be exhaustive or to limit the invention to the exemplary embodiments disclosed. Many modifications and variations are possible in light of the above teachings. It is intended that the scope of the invention be limited not by this detailed description of examples, but rather by the claims appended hereto.

What is claimed is:

1. A method of cleaning a mat, the method comprising:
receiving a user identifier;
receiving the mat;
conveying the mat via a plurality of rollers in response to receiving the user identifier;
illuminating the mat with ultraviolet radiation while conveying the mat;
spraying the mat with a recirculated cleaning solution while conveying the mat;
determining if the recirculated cleaning solution requires refilling using a sensor;
transmitting a signal indicative of a recirculated cleaning solution level; and
rolling up the mat after illuminating and spraying the mat.

2. The method of claim 1, wherein receiving the mat includes facilitating the mat is being inserted in a straight orientation using a presence sensor.

3. The method of claim 1, wherein receiving the mat includes facilitating the mat being inserted in a straight orientation using an activation switch.

4. The method of claim 3, wherein receiving the mat includes facilitating the mat being inserted in a straight orientation using a presence sensor.

5. The method of claim 1, wherein receiving the mat includes facilitating the mat is being inserted in a straight orientation limit switches with gripper wheels on shafts.

6. The method of claim 1, wherein receiving the mat includes guiding the mat with a biased guide plate.

7. The method of claim 1, wherein receiving the mat includes guiding the mat with a belt.

8. The method of claim 7, wherein receiving the mat includes guiding the mat with a biased guide plate.

9. The method of claim 1, wherein receiving the mat includes controlling a motor based on a first sensor and a second different sensor agreeing that the mat is aligned with at least two of the plurality of rollers.

10. The method of claim 1, wherein receiving the mat includes receiving the mat in a rolled state.

11. The method of claim 1, wherein receiving the mat includes receiving the mat in an unrolled state.

12. The method of claim 11, wherein receiving the mat includes receiving the mat in a partially rolled and partially unrolled state.

13. The method of claim 1, wherein conveying the mat includes using a chain and sprocket driven mat conveyance.

14. The method of claim 1, wherein conveying the mat includes conveying the mat using a friction reduction guide plate.

15. The method of claim 1, wherein conveying the mat includes conveying the mat using a horizontal belt tensioner.

16. The method of claim 1, wherein conveying the mat includes dynamically applying a pressure to convey the mat using spring loaded idler roller brackets.

17. The method of claim 1, wherein illuminating the mat with ultraviolet radiation includes only illuminating if an outer cover sensor indicates that an outer cover is closed.

18. The method of claim 1, wherein spraying the mat with a recirculated cleaning solution includes gravity feeding the cleaning solution to a reservoir and pumping the cleaning solution against a gravitational pull.

19. The method of claim 1, wherein spraying the mat includes spraying the mat with an organic cleaning solution.

20. The method of claim 1, wherein spraying the mat includes shielding a flow of cleaning solution.

21. The method of claim 1, wherein determining if the recirculated cleaning solution requires refilling includes using a float switch.

22. The method of claim 1, wherein rolling up the mat includes dynamically adjusting a tension on the mat.

23. The method of claim 22, wherein dynamically adjusting the tension is facilitated by at least one belt and at least one pully.

24. The method of claim 23, wherein conveying the mat employs at least one chain to guide the mat toward the at least one belt and at least one pully at a predetermined speed to roll the mat.

25. The method of claim 1, further comprising automatically unlocking a door after rolling up the mat.

26. The method of claim 1, further comprising automatically opening a door to expose the mat after rolling up the mat.

27. The method of claim 1, further comprising automatically unlocking the door after rolling up the mat.

28. The method of claim 1, further comprising reading a sensor for fault detection when the mat which does not properly complete a cleaning cycle and automatically reversing conveyance of the mat in response to the fault detection.

29. The method of claim 1, further comprising vacuuming a first side of the mat and vacuuming a second different side of the mat.

30. The method of claim 1, wherein rolling up the mat includes using a first spindle and a second opposing spindle.

31. The method of claim 30, wherein using the first spindle includes using a spindle finger with a fist knuckle and a second different knuckle.

32. The method of claim 31, wherein the first knuckle and the second knuckle define a first finger segment, a second finger segment, and a third finger segment.

33. The method of claim 32, including biasing the first finger at a first tension level and biasing the second finger segment at a second different tension level, wherein the first tension level is higher than the second tension level based on the proximity to the a center of the mat.

34. The method of claim 32, including retracting the first spindle away from the mat to enable dropping the mat.

35. The method of claim 30, including sensing a first angle of rotation of the first spindle using a first sensor and sensing a second angle of rotation of the second spindle using a second different sensor.

36. The method of claim 35, wherein the first sensor includes a hall sensor.

37. The method of claim 35, including controlling a motor to keep the first angle of rotation within a predetermined threshold of the second angle of rotation prior to the mat engaging the first spindle.

38. The method of claim 37, wherein the predetermined threshold is less than five angular degrees.

39. The method of claim 35, including controlling a motor to hold the first spindle at a predetermined angle of rotation to facilitate removal by a user.

40. The method of claim 35, including preventing a new mat conveyance based on a diagonal sensor indicating the mat has not been removed from the first spindle.

41. The method of claim 30, including a guide flap biased to guide the mat in to the first spindle.

42. The method of claim 1, including a touch screen display.

43. The method of claim 42, including a user panel, independent of the touch screen display, the user panel including a forward function, a reverse function, and cancel function.
